(19) United States
(12) Patent Application Publication
(10) Pub. No.: US 2014/0323419 A1
(43) Pub. Date: Oct. 30, 2014

(54) HIGH EFFICACY COMBINATION THERAPY FOR HELICOBACTER PYLORI ERADICATION AND PACKAGING FOR THE DISTRIBUTION AND USE THEREOF

(71) Applicant: TAT-KIN TSANG, WILMETTE, IL (US)

(72) Inventor: TAT-KIN TSANG, WILMETTE, IL (US)

(21) Appl. No.: 14/260,508

(22) Filed: Apr. 24, 2014

Related U.S. Application Data
(60) Provisional application No. 61/815,478, filed on Apr. 24, 2013.

(51) Int. Cl. A61J 1/03; A61K 31/7048; A61K 31/43; A61K 31/4439

(52) U.S. Cl. CPC A61J 1/03; A61K 31/4439; A61K 31/7048; A61K 31/43
USPC 514/29; 206/459.5

(57) ABSTRACT

The invention teaches a method for treatment of Helicobacter pylori infection and a packaging system for the distribution, use and implementation of the treatment method.

### CROSS-REFERENCE TO RELATED APPLICATIONS

This US patent application claims the benefit of the filing date of U.S. Provisional Application Ser. No. 61/815,478, filed Apr. 24, 2013, under 35 U.S.C. 119(e).

### BACKGROUND OF THE INVENTION

1. Technical Field

The present disclosure relates to a method for treatment of Helicobacter pylori infection and a packaging system, which simplifies the distribution, use and implementation of the treatment method.

2. Background

Helicobacter pylori is a ubiquitous organism that is present in about 50% of the global population. Chronic infection with H. pylori causes atrophic and even metaplastic changes in the stomach and has a known association with peptic ulcer disease (Santacroce et al., emedicin.medscape.com/article/176938-overview, updated Feb. 15, 2013, retrieved Apr. 17, 2013). Detection of H. Pylori infection can be achieved by various levels of diagnostic testing, starting with a breath test, blood test, or stool test, or more invasively with an endoscopic procedure and pathological biopsy (www.mayoclinic.com/health/h-pylori/DS00958, retrieved Apr. 17, 2013). Chemical assay, such as the CLO (Camplyobactor-like organism) test are used on biopsy samples. Highly sensitive multi-plex PCR assay testing has also been developed for the detection of infection from a variety of tissues.

H. Pylori has been declared by the World Health Organization (WHO) to be a carcinogen for gastric cancer. It is recognized that the incidence of H. pylori infection in different populations varies, and that certain populations are more prone than others to have antibiotic resistant strains to different degree. (F. Megraud, GUT 2004, 53:1374-1384). Antibiotic resistance varies regionally as well. (Wu et al., Gastroenterology Research and Practice 2012, published online Jul. 5, 2012).

Combination therapies for Helicobacter infection of the digestive tract have been developed over the years. Patients, who test positive for H. pylori infection, require sustained treatment and, in order to secure patient compliance, substantial education is necessary. Efficacy of standard therapies and combinations of therapies highlight the need for improved alternative treatments. Triple combination therapies have been reported to achieve cure rates from 85-90% (Santacroce et al., emedicine.medscape.com/article/176938-treatment, updated Feb. 15, 2013, retrieved Apr. 17, 2013).

Some examples of antibiotics used to treat H. pylori are amoxicillin, clarithromycin (BIAXIN™), metronidazole (FLAGYL™) and tetracycline (BRISTACYCLINE™, SUMYCIN™). Prepackaged drug combinations, such as bismuth subcitrate potassium, metronidazole and tetracycline (PYLERA™), bismuth subsalicylate, metronidazole and tetracycline (HELIDAC™), and lansoprazole, amoxicillin and clarithromycin (PREVPAC™), have been used (WebMD www.webmd.corn/digestive-disorders/combination-drug-therapy-for-peptic-ulcer-disease, retrieved Apr. 16, 2013). Each of these therapeutic regimens requires at least twice-a-day dosing.

PREVPAC™ consists of a daily administration card containing two PREVACID™ (lansoprazole) 30 mg capsules, four amoxicillin 500 mg capsules, USP, and two clarithromycin 500 mg tablets, USP, for oral administration. Dosage and administration for H. pylori eradication to reduce the risk of duodenal ulcer recurrence listed in the product information states that the recommended adult oral dosage is 30 mg PREVACID™ (lansoprazole), 1 g amoxicillin, and 500 mg clarithromycin administered together twice daily (morning and evening) for 10 to 14 days. (www.drugs.com/pro/prevpac.html, retrieved Apr. 16, 2013).

A component of the PREVPAC™ is PREVACID™ (lansoprazole) a proton pump inhibitor (PPI). (www.drugs.com/pro/lansoprazole.html, retrieved Apr. 23, 2013). Another component of the PREVPAC™ is BIAXIN™ (clarithromycin), which is an immediate-release formulation of a semi-synthetic macrolide antibiotic. Treatment for H. pylori eradication to reduce the risk of duodenal ulcer recurrence is described in the product information sheet for BIAXIN™.

The triple therapy BIAXIN™/lansoprazole/amoxicillin includes a recommended adult dose of 500 mg BIAXIN™, 30 mg lansoprazole and 1 gram amoxicillin, all given twice daily (q12h) for 10 or 14 days. The triple therapy BIAXIN™/omeprazole/amoxicillin includes a recommended adult dose of 500 mg BIAXIN™, 20 mg omeprazole, and 1 gram amoxicillin, all given twice daily (q12h) for 10 days. In patients with an ulcer present at the time of initiation of therapy, an additional 18 days of omeprazole 20 mg once daily is recommended for ulcer healing and symptom relief.

The dual therapy BIAXIN™/omeprazole includes a recommended adult dose of 500 mg BIAXIN™, given three times daily (q8h) and 40 mg omeprazole given once daily (qAM) for 14 days. An additional 14 days of omeprazole 20 mg once daily is recommended for ulcer healing and symptom relief. The dual therapy BIAXIN™/ranitidine bismuth citrate includes a recommended adult dose of 500 mg BIAXIN™ given twice daily (q12h) or three times daily (q8h) and 400 mg ranitidine bismuth citrate given twice daily (q12h) for 14 days. An additional 14 days of 400 mg twice daily is recommended for ulcer healing and symptom relief (www.drugs.com/pro/biaxin/html, retrieved Apr. 16, 2013).

BIAXIN™ XL is an extended-release formulation of clarithromycin. Extended-release clarithromycin (1000 mg once daily) has been used as an alternative to immediate-release clarithromycin (500 mg twice daily) for the treatment of Helicobacter pylori-associated peptic ulcer disease (Liou et al., Comparative study of modified-release clarithromycin and immediate-release clarithromycin in the treatment of Helicobacter pylori-associated peptic ulcer disease, Hepatogastroenterol. 53(71):792-796 (September-October 2006)). BIAXIN™ XL formulations are described in U.S. Pat. Nos. 6,010,718 and 6,551,616. DEXILANT™ (dexlansoprazole) 60 mg is a delayed-release capsule PPI suitable for once-a-day administration (www.drugs.com/pro/dexilant.html, retrieved Apr. 16, 2013).

DEXILANT™ formulation and use are described in U.S. Pat. Nos. 6,462,058, 6,664,276, 6,939,971, 7,285,668, 7,790,755, 8,105,626, and 8,173,158.

MOXATAG™ (amoxicillin) is an extended-release formulation of amoxicillin intended to provide once-daily dosing available in a 775 mg extended-release formulation (www.drugs.com/pro/moxatag.html, retrieved Apr. 16, 2013). MOXATAG™ formulation and use are disclosed in U.S. Pat. Nos. 6,544,555, 6,669,948, 6,723,341, 8,299,052 and 8,357,394.

The proton pump inhibitor (PPI) omeprazole (sold under the brand name PRILOSEC™) formulated in a delayed-release capsule is indicated in combination therapy for H. pylori eradication for the reduction of the risk of duodenal ulcer recurrence. Triple therapy combines the use of omeprazole delayed-release capsules (20 mg) plus clarithromycin 500 mg plus amoxicillin 1000 mg each given twice daily for 10 days. In patients with ulcer present at the time of initiation of therapy, an additional 18 days of omeprazole delayed-release capsules 20 mg once daily is recommended for ulcer healing and symptom relief.

Dual therapy (Omeprazole/clarithromycin) dosing schedule is given as omeprazole delayed-release capsules 40 mg once daily plus clarithromycin 500 mg three times daily for 14 days. In patients with ulcer present at the time of initiation of therapy, an additional 14 days of omeprazole delayed-release capsules 20 mg once daily is recommended for ulcer healing and symptom relief. Among patients who fail therapy, Omeprazole delayed-release capsules with clarithromycin is more likely to be associated with the development of clarithromycin resistance as compared with triple therapy. In patients who fail therapy, susceptibility testing should be done. If resistance to clarithromycin is demonstrated or susceptibility testing is not possible, alternative antimicrobial therapy should be instituted. (www.drugs.com/pro/omeprazole.html retrieved Apr. 23, 2013).

ZEGERID™ (omeprazole/sodium bicarbonate) is available as a capsule and as a powder for oral suspension in 20 mg and 40 mg strengths of omeprazole for adult use once-a-day to treat gastric ulcer disease. (www.drugs.com/pro/zegerid.html, retrieved Apr. 23, 2013).

ZEGERID™ formulation and use are disclosed in U.S. Pat. Nos. 6,489,346, 6,645,988, 6,699,885, and 7,399,772.

Triple therapy with a proton pump inhibitor (PPI), clarithromycin and amoxicillin or metronidazole given twice daily is the most commonly recommended first choice treatment for Helicobacter pylori infection; however, this treatment only results in an eradication rate of about 85%. Failure to successfully eradicate the H. pylori infection risks enhancing the antibiotic resistance of the infecting bacteria, and increases the difficulty of eradication. Current methods of treatment are complicated by multiple daily dosing, which inhibits and discourages patient compliance and subsequently adversely affects the therapeutic outcomes of such treatment.

Worldwide accepted combination treatments for H. pylori infection are BMT, LAC and OAC. The BMT regimen is based on the administration of bismuth subsalicylate, metronidazole and tetracycline. An H2-receptor agonist is administered for an additional 4 weeks. The LAC regimen is based on the administration of lansoprazole, amoxicillin and clarithromycin. The OAC regimen is based on the administration of omeprazole, amoxicillin and clarithromycin. Increasing resistance to antibiotic treatment has made alternative treatments necessary. Quadruple therapy of omeprazole plus a single 3-in-1 capsule containing bismuth subcitrate potassium, metronidazole and tetracycline has been tested against standard therapy and appears to provide improved eradication (Santacroce et al., emedicine.medscape.com/article/176938-medication, updated Feb. 15, 2013, retrieved Apr. 17, 2013).

### Table 1

Suggested Therapeutic Regimens for Eradication of H. pylori Infection

### First-Line Therapy (7-10 Days)

PPI standard dose twice daily+Clarithromycin 500 mg twice daily+Amoxicillin 1 g twice daily

### Second-Line Therapy (10-14 Days)

PPI standard dose twice daily+Metronidazole 500 mg twice daily or Amoxicillin 1 g twice daily+Tetracycline 4 times daily+Bismuth subcitrate 120 mg 4 times daily

### Rescue Therapies

PPI standard dose twice daily+Rifabutin 300 mg once daily+Amoxicillin 1 g twice daily for 7 days

### Or

PPI standard dose twice daily+Amoxicillin 1 g twice daily+Levofloxacin 500 mg once daily for 7 days

### Or

PPI standard dose twice daily+Amoxicillin 1 g twice daily for 5 days Followed by

PPI standard dose twice daily+Clarithromycin 500 mg twice daily+Tinidazole 500 mg twice daily for 5 days

(Adapted from Romano, Marco and Cuomo, Antonio, Eradication of Helicobacter pylori: A Clinical Update, www.ncbi.nlm.nih.gov/pmc/articles/PMC1140724 published online Feb. 18, 2004, retrieved Apr. 17, 2013)

It is recognized that the difficulty in effectively treating H. pylori infection is impeded by the nature of the organism and the complexity of the current treatment regimens. Impaired patient compliance with complex treatment regimens can contribute to the evolution of greater antibiotic resistant strains in world populations. Antibiotic resistance to the therapeutics selected for various components of combination therapies is known, and continued use of the components with low compliance can result in poor outcomes as well as an increase in antibiotic resistance.

In view of the foregoing, it is desirable to have a treatment therapy for H. pylori infection that offers advantages over currently available treatment therapies. There is a need for improved therapy regimens, and for methods of treatment that enhance patient compliance such as a reduced dosing requirement. Treatment regimens which encourages patient compliance would be an improvement over complex regimens currently used. The ease of use can lead to improved treatment outcome and help reduce increased incidence of antibiotic resistant strains. It is an object of the present disclosure to provide such a treatment therapy and packaging for practicing the methods of the invention. The present invention teaches a treatment regimen and packaging which enables high compliance with the therapy. This and other objects and advantages, as well as inventive features, will become apparent from the detailed description provided herein.

### SUMMARY OF THE INVENTION

The invention provides for:
1. A method of treatment of H. pylori infection in a patient in need thereof, said method comprising the combined use of once-a-day dexlansoprazole, with once-a-day clarithromycin, and once-a-day amoxicillin.
2. A method of treatment of H. pylori infection in a patient in need thereof, said method comprising the combined use of once-a-day dexlansoprazole, with once-a-day clarithromycin, and once-a-day amoxicillin wherein the dose of dexlansoprazole is from about 30 mg to about 60 mg, the dose of clarithromycin is from about 750 mg to about 1500 mg, and the dose of amoxicillin is from about 775 mg to about 2500 mg.
3. A method of treatment of H. pylori infection in a patient in need thereof, said method comprising the combined use of once-a-day dexlansoprazole, with once-a-day clarithromycin, and once-a-day amoxicillin wherein the dose of dexlansoprazole is from about 30 mg to about 60 mg, the dose of clarithromycin is from about 750 mg to about 1500 mg, and the dose of amoxicillin is from about 775 mg to about 2500 mg wherein the dose of dexlansoprazole is about 60 mg, the dose of clarithromycin is about 1000 mg, and the dose of amoxicillin is about 2325 mg.
4. A method of treatment of H. pylori infection in a patient in need thereof, said method comprising the combined use of once-a-day dexlansoprazole, with once-a-day clarithromycin, and once-a-day amoxicillin, wherein said H. pylori is not antibiotic resistant.
5. A method of treatment of H. pylori infection in a patient in need thereof, said method comprising the combined use of once-a-day dexlansoprazole, with once-a-day clarithromycin, and once-a-day amoxicillin, wherein said H. pylori is not antibiotic resistant, wherein said H. pylori is not antibiotic resistant to clarithromycin or amoxicillin.
6. A method of treatment of H. pylori infection in a patient in need thereof, said method comprising the combined use of once-a-day omeprazole, with once-a-day clarithromycin, and once-a-day amoxicillin.
7. A method of treatment of H. pylori infection in a patient in need thereof, said method comprising the combined use of once-a-day omeprazole, with once-a-day clarithromycin, and once-a-day amoxicillin wherein the dose of omeprazole is from about 20 mg to about 40 mg, the dose of clarithromycin is from about 750 mg to about 1500 mg, and the dose of amoxicillin is from about 775 mg to about 2500 mg.
8. A method of treatment of H. pylori infection in a patient in need thereof, said method comprising the combined use of once-a-day omeprazole, with once-a-day clarithromycin, and once-a-day amoxicillin wherein the dose of omeprazole is from about 20 mg to about 40 mg, the dose of clarithromycin is from about 750 mg to about 1500 mg, and the dose of amoxicillin is from about 775 mg to about 2500 mg wherein the dose of omeprazole is about 40 mg, the dose of clarithromycin is about 1000 mg, and the dose of amoxicillin is about 2325 mg.
9. A method of treatment of H. pylori infection in a patient in need thereof, said method comprising the combined use of once-a-day omeprazole, with once-a-day clarithromycin, and once-a-day amoxicillin wherein the dose of omeprazole is from about 20 mg to about 40 mg in the form of an omeprazole/sodium bicarbonate combination, wherein the sodium bicarbonate is present in an amount from about 1 g to about 1.5 g, the dose of clarithromycin is about 1000 mg, and dose of amoxicillin is about 2325 mg.
10. A method of treatment of H. pylori infection in a patient in need thereof, said method comprising the combined use of once-a-day omeprazole, with once-a-day clarithromycin, and once-a-day amoxicillin wherein the dose of omeprazole is from about 20 mg to about 40 mg in the form of an omeprazole/sodium bicarbonate combination, wherein the sodium bicarbonate is present in an amount from about 1 g to about 1.5 g, the dose of clarithromycin is about 1000 mg, and dose of amoxicillin is about 2325 mg wherein the dose of omeprazole is about 40 mg in the form of an omeprazole/sodium bicarbonate combination, wherein the sodium bicarbonate is present in an amount from about 1 g to about 1.5 g, the dose of clarithromycin is about 1000 mg, and dose of amoxicillin is about 2325 mg.
11. A method of treatment of H. pylori infection in a patient in need thereof, said method comprising the combined use of once-a-day omeprazole, with once-a-day clarithromycin, and once-a-day amoxicillin, wherein said H. pylori is not antibiotic resistant.
12. A method of treatment of H. pylori infection in a patient in need thereof, said method comprising the combined use of once-a-day omeprazole, with once-a-day clarithromycin, and once-a-day amoxicillin, wherein said H. pylori is not antibiotic resistant, wherein said H. pylori is not antibiotic resistant to clarithromycin or amoxicillin.
13. A pharmaceutical packaging for use in a method of treatment of H. pylori infection in a patient in need thereof, said method comprising the combined use of once-a-day dexlansoprazole, with once-a-day clarithromycin, and once-a-day amoxicillin, said packaging comprising a single daily dose of a once-a-day dexlansoprazole, a once-a-day clarithromycin, and a once-a-day amoxicillin.
14. A pharmaceutical packaging for use in a method of treatment of H. pylori infection in a patient in need thereof, said method comprising the combined use of once-a-day omeprazole, with once-a-day clarithromycin, and once-a-day amoxicillin, wherein said H. pylori is not antibiotic resistant, said packaging comprising a single daily dose of a once-a-day omeprazole, a once-a-day clarithromycin, and a once-a-day amoxicillin.

15. A kit for the treatment of H. pylori infection in a patient in need thereof, said kit comprising multiple, daily administration packs of a pharmaceutical packaging for use in a method of treatment of H. pylori infection in a patient in need thereof, said method comprising the combined use of once-a-day dexlansoprazole, with once-a-day clarithromycin, and once-a-day amoxicillin, said packaging comprising a single daily dose of a once-a-day dexlansoprazole, a once-a-day clarithromycin, and a once-a-day amoxicillin, in a number sufficient to cover a prescribed course of treatment, and said kit further comprising instructions for use.
16. A kit for the treatment of H. pylori infection in a patient in need thereof, said kit comprising multiple daily administration packs of a pharmaceutical packaging for use in a method of treatment of H. pylori infection in a patient in need thereof, said method comprising the combined use of once-a-day omeprazole, with once-a-day clarithromycin, and once-a-day amoxicillin, wherein said H. pylori is not antibiotic resistant, said packaging comprising a single daily dose of a once-a-day omeprazole, a once-a-day clarithromycin, and a once-a-day amoxicillin, in a number sufficient to cover a prescribed course of treatment, said kit further comprising instructions for use.
17. A pharmaceutical packaging for use in a method of treatment of H. pylori infection in a patient in need thereof, said method comprising the combined use of once-a-day dexlansoprazole, with once-a-day clarithromycin, and once-a-day amoxicillin, wherein said H. pylori is not antibiotic resistant, said packaging comprising a single daily dose of a once-a-day dexlansoprazole, a once-a-day clarithromycin, and a once-a-day amoxicillin.
18. A pharmaceutical packaging for use in a method of treatment of H. pylori infection in a patient in need thereof, said method comprising the combined use of once-a-day omeprazole, with once-a-day clarithromycin, and once-a-day amoxicillin, wherein said H. pylori is not antibiotic resistant, said packaging comprising a single daily dose of a once-a-day omeprazole, a once-a-day clarithromycin, and a once-a-day amoxicillin.
19. A kit for the treatment of H. pylori infection in a patient in need thereof, said kit comprising multiple, daily administration packs of a pharmaceutical packaging for use in a method of treatment of H. pylori infection in a patient in need thereof, said method comprising the combined use of once-a-day dexlansoprazole, with once-a-day clarithromycin, and once-a-day amoxicillin, wherein said H. pylori is not antibiotic resistant, said packaging comprising a single daily dose of a once-a-day dexlansoprazole, a once-a-day clarithromycin, and a once-a-day amoxicillin, in a number sufficient to cover a prescribed course of treatment, and said kit further comprising instructions for use.
20. A kit for the treatment of H. pylori infection in a patient in need thereof, said kit comprising multiple daily administration packs of a pharmaceutical packaging for use in a method of treatment of H. pylori infection in a patient in need thereof, said method comprising the combined use of once-a-day omeprazole, with once-a-day clarithromycin, and once-a-day amoxicillin, wherein said H. pylori is not antibiotic resistant, said packaging comprising a single daily dose of a once-a-day omeprazole, a once-a-day clarithromycin, and a once-a-day amoxicillin, in a number sufficient to cover a prescribed course of treatment, said kit further comprising instructions for use.

### DETAILED DESCRIPTION OF THE INVENTION

The invention teaches a method of treatment for H. pylori infection that encourages patient compliance by providing a once-a-day treatment regimen. The present disclosure provides a method for the combined use of once-a-day dexlansoprazole, with once-a-day clarithromycin, and once-a-day amoxicillin for treatment of H. pylori infection, in a patient in need thereof. Applicant is not aware of such a combination having been taught or suggested in the art, and it is the Applicant's belief that the efficacy of such a combination cannot be predicted based on currently employed therapeutic regimens. The method of the present invention provides for high patient compliance with the treatment regimen. The method of the present invention and the packaging components of the invention enhances patient compliance. Enhanced patient compliance should result in more effective treatment.

The method of treatment encompasses administering a once-a-day formulation of the proton pump inhibitor dexlansoprazole, a once-a-day formulation of amoxicillin and a once-a-day formulation of clarithromycin to a patient in need thereof. The once-a-day formulations are preferably extended-release/delayed-release formulations.

The present disclosure also provides for a combined use of a once-a-day formulation of the proton pump inhibitor omeprazole, a once-a-day formulation of amoxicillin and a once-a-day formulation of clarithromycin for eradication of H. pylori in a patient in need thereof. In a preferred embodiment the proton pump inhibitor is a formulation of omeprazole/sodium bicarbonate.

In a preferred embodiment, the patient to be treated has been identified as being infected with H. pylori. Successful treatment by the method will achieve at least about 80% efficacy in treatment of the H. pylori infection as understood by the standard medical assessments. In one embodiment successful treatment achieves at least about 80% efficacy. In another embodiment, successful treatment achieves at least about 85% efficacy. In another embodiment, successful treatment achieves at least about 90% efficacy.

In the present invention, effective treatment or eradication of H. pylori infection can be achieved with symptomatic relief of the patient. In the present invention, substantial eradication refers to treatment which results in the inability to detect H. pylori infection in a subject per currently practiced diagnostic techniques. It is understood that complete eradication is not required for therapeutic treatment of H. pylori infection.

In a preferred embodiment effective treatment in a non-antibiotic resistant patient population would be achieved with 85% success in achieving therapeutic relief in subject patients. In a preferred embodiment, the patient to be treated has been identified as being infected with H. pylori that is not antibiotic resistant to clarithromycin. In another preferred embodiment, the patient to be treated has been identified as being infected with H. pylori that is not antibiotic resistant to amoxicillin. In another preferred embodiment, the patient to be treated has been identified as being infected with H. pylori that is not antibiotic resistant to both clarithromycin and amoxicillin. Successful treatment by the method wherein the patient to be treated has been identified as being infected with H. pylori that is not antibiotic resistant to both clarithromycin and amoxicillin, will achieve at least about 80% efficacy in treatment of the H. pylori infection as understood by the standard medical assessments. Thus in one embodiment successful treatment achieves at least about 80% efficacy. In another embodiment, successful treatment achieves at least about 85% efficacy. In another embodiment, successful treatment achieves at least about 90% efficacy, wherein the patient to be treated has been identified as being infected with H. pylori that is not antibiotic resistant to both clarithromycin and amoxicillin.

In a preferred embodiment the method of the invention includes administering once-a-day an extended-release/delayed-release dose of from about 30 mg to about 60 mg of dexlansoprazole, such as 30 mg, 35 mg, 40 mg, 45 mg, 50 mg, 55 mg, or 60 mg. In a preferred embodiment the dose of dexlansoprazole is about 60 mg, such as 60 mg.

In another preferred embodiment the method includes administering once-a-day an extended-release/delayed-release dose of from about 20 mg to about 40 mg of omeprazole, such as 20 mg, 25 mg, 30 mg, 35 mg, or 40 mg. In a preferred embodiment the dose of omeprazole is about 40 mg, such as 40 mg.

In another embodiment, the once-a-day formulation of omeprazole is in the form of an omeprazole/sodium bicarbonate combination, wherein the sodium bicarbonate is present in an amount from about 1 g to about 1.5 g, such as 1.0 g, 1.1 g, 1.2 g, 1.3 g, 1.4 g or 1.5 g. In a preferred embodiment the dose of omeprazole/sodium bicarbonate is about 40 mg/1.1 g, such as 40 mg/1.1 g.

In a preferred embodiment the method includes administering once-a-day an extended-release/delayed-release dose from about 750 mg to about 1500 mg of clarithromycin, such as 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg. In a preferred embodiment the dose of clarithromycin is about 1000 mg, such as 1000 mg.

In a preferred embodiment the method includes administering once-a-day an extended-release/delayed-release dose from about 775 mg to about 2500 mg of amoxicillin, such as 775 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg or 2500 mg. In a preferred embodiment the dose of amoxicillin is about 2000 mg, such as 2000 mg. In another embodiment the dose of amoxicillin is about 2325 mg, such as 2325 mg.

In a preferred embodiment the method includes administering the triple combination therapy of a once-a-day formulation of the proton pump inhibitor dexlansoprazole, a once-a-day formulation of amoxicillin and a once-a-day formulation of clarithromycin such that an acceptable effective therapeutic dose is maintained over the course of a day. The method of the present invention envisions the administration of a combined therapy of once-a-day formulations of a proton pump inhibitor, amoxicillin, and clarithromycin to a patient in need of treatment for H. pylori infection for sufficient time so as to achieve symptomatic relief. In a preferred embodiment, treatment following the method of the present invention can be from 1 to 8 weeks. Preferably treatment following the method is for 8 weeks. In another embodiment treatment can be for 7 weeks, 6 weeks, 5 weeks, 4 weeks, 3 weeks, 2 weeks or 1 week, depending upon the individual patient.

The therapeutic regimen includes a course of treatment from around 7 days to around 14 days, such as 7-14 days, 7-13 days, 7-12 days, 7-11 days, or 7-10 days. The length of treatment may be dictated by the initial condition of the patient after screening assessment of the extent of H. pylori infection. A longer course of treatment for greater than 14 days may include anywhere from 15 days to 60 days. A shorter course of treatment, such as less than around 7 days, e.g., 7 days, 6 days, 5 days, 4 days, or 3 days, can be appropriate for lower levels of infection or for maintenance of H. pylori eradication.

The method of the present invention teaches a high efficacy combination therapy that encourages patient compliance. The method of the present invention is suitable for all patients identified with H. pylori infection in need of treatment. The method of the present invention is highly suitable and most highly effective where the strain of H. pylori is not antibiotic resistant to one or more of the treatment therapeutics. In a preferred embodiment, the method of the invention is directed towards treating a patient infected with H. pylori which is not antibiotic resistant to clarithromycin. In another preferred embodiment, the method of the invention is directed towards treating a patient infected with H. pylori which is not antibiotic resistant to amoxicillin. In a most preferred embodiment, the method of the invention is directed towards treating a patient infected with H. pylori which is not antibiotic resistant to clarithromycin or amoxicillin. In a preferred embodiment eradication therapy is conducted for around 14 days, such as 14 days. However, it is possible that a shorter duration of treatment, such as 8, 9, 10, 11, 12, or 13 days of treatment can suffice. In cases of severe infection, a longer course of treatment may be required, such as 14, 15, 16, 17, 18, 19, 20, 21, 22, 23, 24, 25, 26, 27, 28, 29, 30 days or more. Extended treatment may require compliant dosing for about 8 weeks, such as 4, 5, 6, 7 or 8 weeks.

The method of the present invention enable that patient compliance is enhanced over that of other multi drug regimens. It is envisioned that the patient compliance with the treatment methods is greater than about 50% as measured by patient reporting. Compliance with the treatment method of the invention can be greater than 50%. It is envisioned that the treatment method of the present invention can achieve greater than 75% compliance, and in ideal circumstance can be 85% or greater. It is highly desirable to achieve as close to 100% compliance as possible. It is envisioned that compliance as such as 95% can be achieved.

The invention teaches a therapeutic regimen for the treatment of H. pylori infection and high patient compliance with the treatment regimen. High compliance with the methods of the invention enhances high efficacy in treatment. In a preferred embodiment of the invention, the method of the present invention can result in compliance with the treatment regimen, greater than 80% as reported by the patient. It is believed that the method of treatment of the invention can result in better than 80% efficacy, and can achieve better than 90% efficacy in H. pylori treatment and/or eradication in patients who do not have antibiotic resistant strains of H. pylori.

The present invention provides for a method of treatment of H. pylori infection in a patient in need thereof, said method comprising the steps of diagnosing a patient as being infected with H. pylori and needing treatment thereof, prescribing a course of treatment of said infection and administering the treatment, said treatment comprising a combined use of once-a-day dexlansoprazole, with once-a-day clarithromycin, and once-a-day amoxicillin for the duration of the course of treatment, wherein said once-a-day doses are sufficient to maintain therapeutically appropriate levels of active ingredient in the system of the patient, and said course of treatment is of sufficient length to achieve substantial eradication of the H. pylori infection. The method of the invention being such that it encourages patient compliance with administering the treatment.

A required course of treatment can be from 3 days to 60 days and may vary from patient to patient depending upon severity of infection. Ideally it is preferred that treatment is from 1 week to 4 weeks, such as for 1, 2, 3, or 4 weeks. Longer courses of treatment may extend for 5, 6, 7 or 8 weeks or longer as medically required.

Therapeutically effective treatment of the method of the invention teaches administering, once-a-day an extended-release/delayed-release dose of from about 30 mg to about 60 mg of dexlansoprazole, such as 30 mg, 35 mg, 40 mg, 45 mg, 50 mg, 55 mg, or 60 mg. In a preferred embodiment the dose of dexlansoprazole is about 60 mg, such as 60 mg. In combination with administering once-a-day an extended-release/delayed-release dose from about 750 mg to about 1500 mg of clarithromycin, such as 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg. In a preferred embodiment the dose of clarithromycin is about 1000 mg, such as 1000 mg. In combination with administering once-a-day an extended-release/delayed-release dose from about 775 mg to about 2500 mg of amoxicillin, such as 775 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg or 2500 mg. In a preferred embodiment the dose of amoxicillin is about 2000 mg, such as 2000 mg. In another embodiment the dose of amoxicillin is about 2325 mg, such as 2325 mg.

The present invention also provides for a method of treatment for H. pylori infection in a patient in need thereof, said method comprising the steps of diagnosing a patient as being infected with H. pylori and needing treatment thereof, prescribing a course of treatment for said infection and administering the treatment, said treatment comprising a combined use of once-a-day omeprazole, with once-a-day clarithromycin, and once-a-day amoxicillin for the duration of the course of treatment, wherein said once-a-day doses are sufficient to maintain therapeutically appropriate levels of active ingredient in the system of the patient, and said course of treatment is of sufficient length to achieve substantial eradication of the H. pylori infection. The method of the invention being such that it encourages patient compliance with administering the treatment.

A required course of treatment can be from 3 days to 60 days and may vary from patient to patient depending upon severity of infection. Ideally it is preferred that treatment is from 1 week to 4 weeks, such as for 1, 2, 3, or 4 weeks. Longer courses of treatment may extend for 5, 6, 7 or 8 weeks or longer as medically required.

Therapeutically effective treatment of the method of the invention teaches administering a once-a-day an extended-release/delayed-release dose of from about 20 mg to about 40 mg of omeprazole, such as 20 mg, 25 mg, 30 mg, 35 mg, or 40 mg. In a preferred embodiment the dose of omeprazole is about 40 mg, such as 40 mg. In combination with a once-a-day an extended-release/delayed-release dose from about 750 mg to about 1500 mg of clarithromycin, such as 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg. In a preferred embodiment the dose of clarithromycin is about 1000 mg, such as 1000 mg. In combination with a once-a-day an extended-release/delayed-release dose from about 775 mg to about 2500 mg of amoxicillin, such as 775 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg or 2500 mg. In a preferred embodiment the dose of amoxicillin is about 2000 mg, such as 2000 mg. In another embodiment the dose of amoxicillin is about 2325 mg, such as 2325 mg.

The present invention also provides for a method of treatment for H. pylori infection in a patient in need thereof, said method comprising the steps of diagnosing a patient as being infected with H. pylori and needing treatment thereof, prescribing a course of treatment for said infection and administering the treatment, said treatment comprising a combined use of once-a-day omeprazole/sodium bicarbonate, with once-a-day clarithromycin, and once-a-day amoxicillin for the duration of the course of treatment, wherein said once-a-day doses are sufficient to maintain therapeutically appropriate levels of active ingredient in the system of the patient, and said course of treatment is of sufficient length to achieve substantial eradication of the H. pylori infection. The method of the invention being such that it encourages patient compliance with administering the treatment.

A required course of treatment can be from 3 days to 60 days and may vary from patient to patient depending upon severity of infection. Ideally it is preferred that treatment is from 1 week to 4 weeks, such as for 1, 2, 3, or 4 weeks. Longer courses of treatment may extend for 5, 6, 7 or 8 weeks or longer as medically required.

Therapeutically effective treatment of the method of the invention teaches administering a once-a-day formulation of omeprazole that is in the form of an omeprazole/sodium bicarbonate combination, having from about 20 mg to about 40 mg of omeprazole, such as 20 mg, 25 mg, 30 mg, 35 mg, or 40 mg. In a preferred embodiment the dose of omeprazole is about 40 mg, such as 40 mg, wherein the sodium bicarbonate is present in an amount from about 1 g to about 1.5 g, such as 1.0 g, 1.1 g, 1.2 g, 1.3 g, 1.4 g or 1.5 g. In a preferred embodiment the dose of omeprazole/sodium bicarbonate is about 40 mg/1.1 g, such as 40 mg/1.1 g. In combination with a once-a-day an extended-release/delayed-release dose from about 750 mg to about 1500 mg of clarithromycin, such as 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg. In a preferred embodiment the dose of clarithromycin is about 1000 mg, such as 1000 mg. In combination with a once-a-day an extended-release/delayed-release dose from about 775 mg to about 2500 mg of amoxicillin, such as 775 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg or 2500 mg. In a preferred embodiment the dose of amoxicillin is about 2000 mg, such as 2000 mg. In another embodiment the dose of amoxicillin is about 2325 mg, such as 2325 mg.

The treatment method of the present invention allows for convenient use and improved patient compliance in using the therapeutics administered, and for high efficacy of therapeutic relief from symptoms of H. pylori infection, eradication of infection, and maintenance of patient status.

The present invention also provides for a pharmaceutical packaging for use in a method of treatment of H. pylori infection in a patient in need thereof, said packaging comprising a single days dose of a once-a-day dexlansoprazole, with a single days dose of a once-a-day clarithromycin, and a single days dose of a once-a-day amoxicillin. The packaging would comprise therapeutically appropriate dose combinations as described for the method of the invention.

The present invention also provides for a pharmaceutical packaging for use in a method of treatment of H. pylori infection in a patient in need thereof, said packaging comprising a single days dose of a once-a-day omeprazole, with a single days dose of a once-a-day clarithromycin, and a single days dose of a once-a-day amoxicillin. The packaging would comprise therapeutically appropriate dose combinations as described for the method of the invention.

The present invention also provides for a pharmaceutical packaging for use in a method of treatment of H. pylori infection in a patient in need thereof, said packaging comprising a single days dose of a once-a-day omeprazole/sodium bicarbonate, with a single days dose of a once-a-day clarithromycin, and a single days dose of a once-a-day amoxicillin. The packaging would comprise therapeutically appropriate dose combinations as described for the method of the invention.

The present invention also provides for a kit for the treatment of H. pylori infection, in a patient in need thereof, said kit comprising multiple, daily administration packs in a number sufficient to cover a prescribed course of treatment, said daily administration packs each comprising a single days dose of a once-a-day dexlansoprazole, with a single days dose of a once-a-day clarithromycin, and a single days dose of a once-a-day amoxicillin, and said kit further comprising instructions for use. The daily administration packs would comprise therapeutically appropriate dose combinations as described for the method of the invention.

The present invention also provides for a kit for the treatment of H. pylori infection, in a patient in need thereof, said kit comprising multiple, daily administration packs in a number sufficient to cover a prescribed course of treatment, said daily administration packs each comprising a single days dose of a once-a-day omeprazole, with a single days dose of a once-a-day clarithromycin, and a single days dose of a once-a-day amoxicillin, and said kit further comprising instructions for use. The daily administration packs would comprise therapeutically appropriate dose combinations as described for the method of the invention.

The present invention also provides for a kit for the treatment of H. pylori infection, in a patient in need thereof, said kit comprising multiple, daily administration packs in a number sufficient to cover a prescribed course of treatment, said daily administration packs each comprising a single days dose of a once-a-day omeprazole/sodium bicarbonate, with a single days dose of a once-a-day clarithromycin, and a single days dose of a once-a-day amoxicillin, and said kit further comprising instructions for use. The daily administration packs would comprise therapeutically appropriate dose combinations as described for the method of the invention.

The present invention also provides for a method of treatment of H. pylori infection in a patient in need thereof, where the H. pylori is not antibiotic resistant, said method comprising the steps of diagnosing a patient as being infected with H. pylori and needing treatment thereof, prescribing a course of treatment of said infection and administering the treatment, said treatment comprising a combined use of once-a-day dexlansoprazole, with once-a-day clarithromycin, and once-a-day amoxicillin for the duration of the course of treatment, wherein said once-a-day doses are sufficient to maintain therapeutically appropriate levels of active ingredient in the system of the patient, and said course of treatment is of sufficient length to achieve substantial eradication of the H. pylori infection. The method of the invention being such that it encourages patient compliance with administering the treatment.

A required course of treatment can be from 3 days to 60 days and may vary from patient to patient depending upon severity of infection. Ideally it is preferred that treatment is from 1 week to 4 weeks, such as for 1, 2, 3, or 4 weeks. Longer courses of treatment may extend for 5, 6, 7 or 8 weeks or longer as medically required.

Therapeutically effective treatment of the method of the invention teaches administering, once-a-day an extended-release/delayed-release dose of from about 30 mg to about 60 mg of dexlansoprazole, such as 30 mg, 35 mg, 40 mg, 45 mg, 50 mg, 55 mg, or 60 mg. In a preferred embodiment the dose of dexlansoprazole is about 60 mg, such as 60 mg. In combination with administering once-a-day an extended-release/delayed-release dose from about 750 mg to about 1500 mg of clarithromycin, such as 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg. In a preferred embodiment the dose of clarithromycin is about 1000 mg, such as 1000 mg. In combination with administering once-a-day an extended-release/delayed-release dose from about 775 mg to about 2500 mg of amoxicillin, such as 775 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg or 2500 mg. In a preferred embodiment the dose of amoxicillin is about 2000 mg, such as 2000 mg. In another embodiment the dose of amoxicillin is about 2325 mg, such as 2325 mg.

The present invention also provides for a pharmaceutical packaging for use in a method of treatment of H. pylori infection in a patient in need thereof, where said H. pylori is not antibiotic resistant to one of the packaging components, said packaging comprising a single days dose of a once-a-day dexlansoprazole, with a single days dose of a once-a-day clarithromycin, and a single days dose of a once-a-day amoxicillin. The packaging would comprise therapeutically appropriate dose combinations as described for the method of the invention.

The present invention also provides for a kit for the treatment of H. pylori infection, in a patient in need thereof, where said H. pylori is not antibiotic resistant to one of the kit components, said kit comprising multiple, daily administration packs in a number sufficient to cover a prescribed course of treatment, said daily administration packs each comprising a single days dose of a once-a-day dexlansoprazole, with a single days dose of a once-a-day clarithromycin, and a single days dose of a once-a-day amoxicillin, and said kit further comprising instructions for use. The daily administration packs would comprise therapeutically appropriate dose combinations as described for the method of the invention.

The pharmaceutical packaging and kits of the present invention allow for convenient use and improved patient compliance. The pharmaceutical packaging and kits of the present invention also allow for efficient and varied packaging, distribution and dispensing of therapeutics for administration and practicing the method of the present invention.

The pharmaceutical packaging and kits of the present invention allow for convenient use and improved patient compliance in using the method of the present invention, and for high efficacy of therapeutic relief from symptoms of H. pylori infection, eradication of infection, and maintenance of patient status.

Also provided is an article of manufacture, such as an article of manufacture which promotes the use of the therapeutic method. In one embodiment, an article of manufacture is a pharmaceutically acceptable package, which presents a single daily administration of each of the active agents. The package can be in the form of a bottle, card, blister pack, box, bag, and the like, which are readily known in the art. The daily administration pack would comprise therapeutically appropriate dose combinations as described for the method of the invention. The daily administration pack may have individually packaged doses in one or more form, such that the sum of the dosage forms are the appropriate dose as taught by the method of the invention. The daily administration pack may be a combination of separately packaged doses, combined, or such combinations as would be efficient for manufacture and distribution. Thus the daily package could be a container which contains all prescribed medicines in a single or multiple compartment packaging. Alternatively, the daily package could be a container which contains multiple sub-packages, each of which containing one or more dosage form, such that the sum total of the daily package comprises a daily therapeutic treatment dose as taught by the method of the invention.

The daily package comprises a once-a-day formulation of the proton pump inhibitor dexlansoprazole, a once-a-day formulation of amoxicillin and a once-a-day formulation of clarithromycin. In another embodiment of the invention, the daily package contains a once-a-day formulation of the proton pump inhibitor omeprazole, a once-a-day formulation of amoxicillin and a once-a-day formulation of clarithromycin. In a further embodiment of the invention, the daily package contains a once-a-day formulation of omeprazole/sodium bicarbonate, a once-a-day formulation of amoxicillin and a once-a-day formulation of clarithromycin.

It is envisioned that these components can be in the form of tablets, capsules, pills, or orally-disintegrable formulas, and of sufficient number to provide the prescribed therapeutic dose. The daily package can further comprise instructions, such as written or illustrated instructions, that describe the method of treatment. In this regard, the instructions can be incorporated into the package, itself, such as by labeling of blisters in a blister pack, for example.

In another embodiment, the single daily package is further packaged, bundled or distributed as a kit of multiple, daily packages sufficient to encompass the prescribed course of treatment. In a preferred embodiment, such a kit contains sufficient daily packages to allow a course of treatment from about 3 days to about 14 days, such as 3 days to 14 days. In another embodiment of the invention, the kit contains sufficient daily packages to allow for a course of treatment from 7 to 14 days, such as 7 days to 14 days. In a preferred embodiment the kit contains sufficient daily packages to allow a course of treatment for about 10 days, such as 10 days. In another preferred embodiment the kit contains enough daily packages for a course of treatment of about 14 days, such as 14 days. Thus permutations of kits could be provided which encompass anywhere from 3 days to 60 days of treatment or more sufficient to achieve effective treatment. In a further embodiment of the present invention, a multi-day kit is packaged with instructions describing the method of treatment.

Methods for diagnosing a patient as being infected with H. pylori are known in the art, and ascertained by accepted standards. In a preferred embodiment, infection can be ascertained by breath test. As medically appropriate, confirmation with PCR can be optionally conducted.

The composition of once-a-day doses which are sufficient to maintain therapeutically appropriate levels of active ingredient in the system of the patient, can be determined by appropriate pharmacological testing. It is acceptable to use published AUC (area under the curve) data to ascertain dosing requirements.

Successful treatment for the substantial eradication of H. pylori infection in a patient in need thereof, can be measured by acceptable medical testing. In a preferred embodiment, substantial eradication can be ascertained by a negative breath test.

### EXAMPLES

The following examples serve to illustrate the present invention. The examples are not intended to limit the scope of the claimed invention in any way.

### Example 1

This example describes demonstrating the therapeutic efficacy of the method of the present disclosure through the use of daily administration of a once-a-day formulation of the proton pump inhibitor dexlansoprazole (60 mg qd), a once-a-day formulation of amoxicillin (2325 mg qd) and a once-a-day formulation of clarithromycin (1000 mg qd). About 30 patients with endoscopically proven positive Helicobacter pylori infection are recruited into the study. Primary care physicians as well as gastroenterologists are requested for patient referral for the study.

Diagnosis of Helicobacter pylori infection is made by positive histology and/or positive rapid urease test. Patients with previous Helicobacter pylori treatment, pregnancy, lactation, previous gastric surgery, malignancy, major systemic illness or allergy to any one of the medication in the treatment regimen are excluded from the study.

Patients enrolled in the study receive dexlansoprazole 60 mg, clarithromycin extended-release 1000 mg and amoxicillin extended-release 2325 mg once a day for 14 days. During the treatment period, patients are instructed to keep a diary to monitor compliance and side effects.

Six weeks after treatment, Helicobacter pylori eradication is assessed by standard testing methods, including by negative histology and/or rapid urease test and/or negative HP MPCR (H. pylori multiplex Polymerase Chain Reaction), and/or negative HP (H. pylori) breath test, and/or negative HP (H. pylori) stool antigen test.

### Example 2

This example describes demonstrating the comparative therapeutic efficacy of the method of the present disclosure through the use of daily administration of a once-a-day formulation of the proton pump inhibitor dexlansoprazole (60 mg qd), a once-a-day formulation of amoxicillin (2325 mg qd) and a once-a-day formulation of clarithromycin (1000 mg qd) and in comparison with the currently employed LAC regimen of lansoprazole (30 mg bid), amoxicillin (1000 mg bid) and clarithromycin (500 mg bid). About 30 patients with endoscopically proven Helicobacter pylori positive functional dyspepsia are recruited into the study. Primary care physicians as well as gastroenterologists are requested for patient referral for the study.

Diagnosis of Helicobacter pylori infection will be made by positive histology and/or positive rapid urease test. Patients with peptic ulcer, previous Helicobacter pylori treatment, pregnancy, lactation, previous gastric surgery, malignancy, major systemic illness or allergy to any one of the medication in the treatment regimen are excluded from the study.

A written informed consent is obtained from all enrolled patients.

Patients enrolled in the study are randomly assigned into two treatment groups. One group receives lansoprazole 30 mg twice daily, clarithromycin 500 mg and amoxicillin 1000 mg twice a day for 14 days. Another group receives dexlansoprazole 60 mg, clarithromycin extended release 1000 mg and amoxicillin extended release 2325 mg once a day for 14 days. During the treatment period, patients are instructed to keep a diary to monitor compliance and side effects.

Six weeks after treatment, Helicobacter pylori eradication is assessed by standard testing methods, including by negative histology and/or rapid urease test and/or negative HP MPCR, and/or negative HP breath test, and/or negative HP stool antigen test. Statistical analysis of Intention-to-treat (ITT) and per protocol (PP) analyses are used to assess the eradication rates of Helicobacter pylori infection in the two groups. The eradication rates and frequencies of adverse effects are compared using the Chi-squared test.

### Example 3

This example describes demonstrating the therapeutic efficacy of the method of the present invention through the use of daily administration of a once-a-day formulation of the proton pump inhibitor dexlansoprazole (60 mg qd), a once-a-day formulation of amoxicillin (1550 mg qd) and a once-a-day formulation of clarithromycin (1000 mg qd). About 30 patients with endoscopically proven positive Helicobacter pylori infection are recruited into the study. Primary care physicians as well as gastroenterologists are requested for patient referral for the study.

Diagnosis of Helicobacter pylori infection is made by positive histology and/or positive rapid urease test. Patients with previous Helicobacter pylori treatment, pregnancy, lactation, previous gastric surgery, malignancy, major systemic illness or allergy to any one of the medication in the treatment regimen are excluded from the study.

Patients enrolled in the study receive dexlansoprazole 60 mg, clarithromycin extended release 1000 mg and amoxicillin extended release 1550 mg once a day for 14 days. During the treatment period, patients are instructed to keep a diary to monitor compliance and side effects.

Six weeks after treatment, Helicobacter pylori eradication is assessed by standard testing methods, including by negative histology and/or rapid urease test and/or negative HP MPCR, and/or negative HP breath test, and/or negative HP stool antigen test.

### Example 4

This example describes demonstrating the therapeutic efficacy of the method of the present disclosure through the use of daily administration of a once-a-day formulation of the proton pump inhibitor dexlansoprazole (60 mg qd), a once-a-day formulation of amoxicillin (2000 mg qd) and a once-a-day formulation of clarithromycin (1000 mg qd). At least 30 patients with endoscopically proven positive Helicobacter pylori infection are recruited into the study. Primary care physicians as well as gastroenterologists are requested for patient referral for the study.

Diagnosis of Helicobacter pylori infection is made by positive histology and/or positive rapid urease test. Patients with previous Helicobacter pylori treatment, pregnancy, lactation, previous gastric surgery, malignancy, major systemic illness or allergy to any one of the medication in the treatment regimen are excluded from the study.

Patients enrolled in the study receive dexlansoprazole 60 mg, clarithromycin extended release 1000 mg and amoxicillin extended release 2000 mg once a day for 14 days. During the treatment period, patients are instructed to keep a diary to monitor compliance and side effects.

Six weeks after treatment, Helicobacter pylori eradication is assessed by standard testing methods, including by negative histology and/or rapid urease test and/or negative HP MPCR, and/or negative HP breath test, and/or negative HP stool antigen test.

### Example 5

This example describes demonstrating the therapeutic efficacy of the method of the present disclosure through the use of daily administration of a once-a-day formulation of the proton pump inhibitor omeprazole/sodium bicarbonate (40 mg/1.1 g qd), a once-a-day formulation of amoxicillin (2325 mg qd) and a once-a-day formulation of clarithromycin (1000 mg qd).

About 30 patients with endoscopically proven positive Helicobacter pylori infection are recruited into the study. Primary care physicians as well as gastroenterologists are requested for patient referral for the study.

Diagnosis of Helicobacter pylori infection is made by positive histology and/or positive rapid urease test. Patients with previous Helicobacter pylori treatment, pregnancy, lactation, previous gastric surgery, malignancy, major systemic illness or allergy to any one of the medication in the treatment regimen are excluded from the study.

Patients enrolled in the study receive omeprazole/sodium bicarbonate 40 mg/1.1 g, clarithromycin extended-release 1000 mg and amoxicillin extended-release 2325 mg once a day for 14 days. During the treatment period, patients are instructed to keep a diary to monitor compliance and side effects.

Six weeks after treatment, Helicobacter pylori eradication is assessed by standard testing methods, including by negative histology and/or rapid urease test and/or negative HP MPCR, and/or negative HP breath test, and/or negative HP stool antigen test.

### Example 6

This example describes a packaging system for the distribution of and/or use of a treatment regimen of the present disclosure.

In a preferred embodiment the packaging system provides for a conveniently distributed and easily used daily therapeutic regimen comprising once-a-day (qd) formulations of dexlansoprazole with once-a-day (qd) amoxicillin and once-a-day (qd) clarithromycin.

In another embodiment the packaging system provides for a conveniently distributed and easily used daily therapeutic regimen comprising once-a-day (qd) formulations of omeprazole with once-a-day (qd) amoxicillin and once-a-day (qd) clarithromycin.

In yet another embodiment the packaging system provides for a conveniently distributed and easily used daily therapeutic regimen comprising once-a-day (qd) formulations of omeprazole/sodium bicarbonate with once-a-day (qd) amoxicillin and once-a-day (qd) clarithromycin.

It is contemplated that the individual daily packages can be prescribed individually or in kits, which combine a sufficient number of daily packs to encompass the full course of treatment. For example, depending upon the clinical presentation of the patient, it may be desirable to prescribe a course of treatment according to the present invention of as few as 7 days, or as many as 60 days or longer. Individual daily packs corresponding to the prescribed days of treatment can thus be provided either individually or in pre-packaged kits.

The composition of the individual daily pack may take many forms and combine any number of individual pharmaceutical dosage units, such as pills, capsules, tablets, orally-disintegrable tablets and the like, the essential feature being that the daily pack provides a single day's dosage of the once-a-day (qd) formulation of dexlansoprazole or omeprazole with the once-a-day (qd) formulation of amoxicillin and the once-a-day (qd) formulation of clarithromycin.

In a preferred embodiment the composition of the individual daily pack provides for a single day's dose of the once-a-day (qd) formulation of dexlansoprazole with the once-a-day (qd) formulation of amoxicillin and the once-a-day (qd) formulation of clarithromycin. In another preferred embodiment the composition of the individual daily pack provides for a single day's dose of the once-a-day (qd) formulation of omeprazole or omeprazole/sodium bicarbonate with the once-a-day (qd) formulation of amoxicillin and the once-a-day (qd) formulation of clarithromycin.

The format of the individual daily pack may also take on many forms and encompass such safety features as deemed appropriate, e.g., bottles, bubble packs, sealed cards, packets and the like, the essential feature of the format of the individual daily pack being that it provides for a single day's dosage of the once-a-day (qd) formulation of dexlansoprazole or omeprazole with the once-a-day (qd) formulation of amoxicillin and the once-a-day (qd) formulation of clarithromycin.

In a preferred embodiment the format of the individual daily pack provides for a single day's dose of the once-a-day (qd) formulation of dexlansoprazole with the once-a-day (qd) formulation of amoxicillin and the once-a-day (qd) formulation of clarithromycin. In another preferred embodiment the format of the individual daily pack provides for a single day's dose of the once-a-day (qd) formulation of omeprazole or omeprazole/sodium bicarbonate with the once-a-day (qd) formulation of amoxicillin and the once-a-day (qd) formulation of clarithromycin.

### Example 7

The method of the invention allows for the manufacture and distribution of kits that will enhance delivery and distribution, as well as enhance patient compliance.

It is contemplated that there can be kits for the practice of the methods of the invention which combine individual daily doses of each therapeutic agent, where each therapeutic agent is packaged separately from different therapeutic agents, but these segregated packages are combined in a larger package or container. For instance, individual bottles, shrink wraps, blister packs or the like could be made up with an individual or multiple daily doses of a single therapeutic agent, then combined together into a kit of the present invention.

Instructions for use included with the kit could provide, for example, product information and dosing.

The packaging of the kit can be a box, container, or other such solid semi-rigid or rigid containment vessel made from any suitable material. Alternatively the packaging can be a bag, shrink-wrap, envelope or any other such flexible container.

It is therefore possible to deliver therapeutic product, individually packaged as a once-a-day unit comprising the appropriate dose of all therapeutic products to be administered on any given day of the treatment. Upon distribution, or pre-packaged, an appropriate number of single day dosing of therapeutics are collected together in a kit packaging for dispensing to the patient to be treated.

Alternatively, it is also possible to deliver therapeutic product, individually packaged as a once-a-day unit comprising the appropriate dose of one therapeutic product to be administered on any given day of the treatment. Upon distribution, or pre-packaged, an appropriate number of single day dosing of all the therapeutics are collected together in a kit packaging for dispensing to the patient to be treated.

### Example 8

Five subjects (4 males and 1 female; 3 Asians and 2 Caucasians; age 34-62 with a mean of 48.6) were identified with conditions requiring therapeutic treatment by the methods of the present invention, and/or with articles of manufacture of the present invention. Informed consent was obtained. Two of the subjects were diagnosed using H. pylori (HP) breath tests, the other three with CLO testing. Of the three undergoing CLO testing under endoscopy, one was confirmed with pathology, one was confirmed with HP-MPCR, and one with both pathology and HP-MPCR.

Each subject was given a regimen of treatment which combined dexlansoprazole (DEXILANT™) 60 mg/capsule one capsule PO (total dose 60 mg), clarithromycin extended release (BIAXIN™ XL) 500 mg/tablet two tablets PO (total dose 1000 mg), and amoxicillin extended release (MOXATAG™) 775 mg/tablets three tablets PO (total dose 2325 mg). This combination therapy was administered each morning, half an hour before breakfast for 14 days.

Upon completion of the therapy, the patients were cleansed for four weeks without treatment. The subjects were then re-tested.

The compliance rate was reported as 100% by the subjects. Four of the five subjects (80%) responded to the treatment. Three had negative HP breath test. More stringent testing with one subject found negative CLO test, and HP-MPCR.

The one subject who did not achieve complete clearance was an Asian, and had a positive breath test. It is known that Asian populations have a higher incident of antibiotic resistant strain of H. pylori in general. With subsequent treatment with bismuth subcitrate potassium (PYLERA™) 3 capsules qid (four times daily) for 10 days, the subject was then retested with negative breath test. This result indicates that this patient was infected with an antibiotic resistant strain.

Thus, 100% of patients who were not antibiotic resistant were effectively treated with the original course of treatment.

TABLE 2
RESULTS

| | | | | | | Post | |
|---|---|---|---|---|---|---|---|
| | | | Method of | | | TREATMENT | |
| ID | AGE | SEX | Diagnosis | Treatment | COMPLIANCE | DIAGNOSIS | COMMENTS |
| 1 | 34 | M | +BREATH | Q day | 100% | −BREATH | SYMPTOM |
| | | | TEST | Clarithro.XL | | TEST | EGD--PATH |
| | | | | Amox XL | | | −HP MPCR |
| | | | | Dexlan 60 | | | |
| 2 | 37 | M | +CLO | SAME | 100% | −Breath | |
| | | | +HP MPCR | | | test | |
| 3 | 50 | M | +PATH, CLO | SAME | 100% | +BREATH | TREATED- |
| | | | +HP MPCR | | | TEST | Additional 10 |
| | | | | | | | days (qid bsp) |
| | | | | | | | −BREATH |
| | | | | | | | TEST |
| 4 | 60 | F | +PATH, CLO | SAME | 100% | −BREATH | |
| | | | +HP MPCR | | | TEST | |
| 5 | 62 | M | +BREATH | SAME | 100% | −PATH, CLO | |
| | | | TEST | | | −HP MPCR | |

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation.

Any trademarks referenced herein are the property of their respective owners.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as defined by the appended claims.

What is claimed is:

1. A method of treatment of H. pylori infection in a patient in need thereof, said method comprising the combined use of once-a-day dexlansoprazole, with once-a-day clarithromycin, and once-a-day amoxicillin.

2. The method of claim 1 wherein the dose of dexlansoprazole is from about 30 mg to about 60 mg, the dose of clarithromycin is from about 750 mg to about 1500 mg, and the dose of amoxicillin is from about 775 mg to about 2500 mg.

3. The method of claim 2 wherein the dose of dexlansoprazole is about 60 mg, the dose of clarithromycin is about 1000 mg, and the dose of amoxicillin is about 2325 mg.

4. The method of claim 1, wherein said H. pylori is not antibiotic resistant.

5. The method of claim 4, wherein said H. pylori is not antibiotic resistant to clarithromycin or amoxicillin.

6. A method of treatment of H. pylori infection in a patient in need thereof, said method comprising the combined use of once-a-day omeprazole, with once-a-day clarithromycin, and once-a-day amoxicillin.

7. The method of claim 6 wherein the dose of omeprazole is from about 20 mg to about 40 mg, the dose of clarithromycin is from about 750 mg to about 1500 mg, and the dose of amoxicillin is from about 775 mg to about 2500 mg.

8. The method of claim 7 wherein the dose of omeprazole is about 40 mg, the dose of clarithromycin is about 1000 mg, and the dose of amoxicillin is about 2325 mg.

9. The method of claim 6 wherein the dose of omeprazole is from about 20 mg to about 40 mg in the form of an omeprazole/sodium bicarbonate combination, wherein the sodium bicarbonate is present in an amount from about 1 g to about 1.5 g, the dose of clarithromycin is about 1000 mg, and dose of amoxicillin is about 2325 mg.

10. The method of claim 9 wherein the dose of omeprazole is about 40 mg in the form of an omeprazole/sodium bicarbonate combination, wherein the sodium bicarbonate is present in an amount of about 1.1 g, the dose of clarithromycin is about 1000 mg, and dose of amoxicillin is about 2325 mg.

11. A method of claim 6, wherein said H. pylori is not antibiotic resistant.

12. A method of claim 11, wherein said H. pylori is not antibiotic resistant to clarithromycin or amoxicillin.

13. A pharmaceutical packaging for use in a method of treatment of claim 1, said packaging comprising a single daily dose of a once-a-day dexlansoprazole, a once-a-day clarithromycin, and a once-a-day amoxicillin.

14. A pharmaceutical packaging for use in a method of treatment of claim 6, said packaging comprising a single daily dose of a once-a-day omeprazole, a once-a-day clarithromycin, and a once-a-day amoxicillin.

15. A kit for the treatment of H. pylori infection in a patient in need thereof, said kit comprising multiple, daily administration packs of a pharmaceutical packaging of claim 13, in a number sufficient to cover a prescribed course of treatment, and said kit further comprising instructions for use.

16. A kit for the treatment of H. pylori infection in a patient in need thereof, said kit comprising multiple daily administration packs of a pharmaceutical packaging of claim 14, in a number sufficient to cover a prescribed course of treatment, said kit further comprising instructions for use.

17. A pharmaceutical packaging for use in a method of treatment of claim 4, said packaging comprising a single daily dose of a once-a-day dexlansoprazole, a once-a-day clarithromycin, and a once-a-day amoxicillin.

18. A pharmaceutical packaging for use in a method of treatment of claim 11, said packaging comprising a single daily dose of a once-a-day omeprazole, a once-a-day clarithromycin, and a once-a-day amoxicillin.

19. A kit for the treatment of H. pylori infection in a patient in need thereof, said kit comprising multiple, daily administration packs of a pharmaceutical packaging of claim 17, in a number sufficient to cover a prescribed course of treatment, and said kit further comprising instructions for use.

20. A kit for the treatment of H. pylori infection in a patient in need thereof, said kit comprising multiple daily administration packs of a pharmaceutical packaging of claim 18, in a number sufficient to cover a prescribed course of treatment, said kit further comprising instructions for use.
